(19) United States
(12) Patent Application Publication
(10) Pub. No.: US 2016/0136285 A1
(43) Pub. Date: May 19, 2016

(54) AN ISOLATED IMMUNOGENIC BACTERIAL ANTIGEN AND ITS USE IN THE PREVENTION AND TREATMENT OF INFECTIONS CAUSED BY GRAM-NEGATIVE BACTERIA

(71) Applicant: WROCLAWSKIE CENTRUM BADAN EIT+ SP. Z O.O., Wroclaw (PL)

(72) Inventors: Tomasz GOZDZIEWICZ, Wroclaw (PL); Jolanta LUKASIEWICZ, Wroclaw (PL); Czeslaw LUGOWSKI, Wroclaw (PL)

(73) Assignee: WROCLAWSKIE CENTRUM BADAN EIT+ SP. Z O.O., Wroclaw (PL)

(21) Appl. No.: 14/896,198

(22) PCT Filed: Jun. 4, 2014
(86) PCT No.: PCT/IB2014/061943

§ 371 Date: Dec. 4, 2015

(30) Foreign Application Priority Data
Jun. 6, 2013 (PL) P.404229

(51) Int. Cl. A61K 47/48; A61K 39/39; A61K 39/02; C07K 16/12; A61K 39/112; A61K 39/108

(52) U.S. Cl. CPC A61K 47/4823; A61K 39/0283; A61K 39/0275; A61K 39/0266; A61K 39/025; A61K 39/0258; C07K 16/1228; C07K 16/1232; C07K 16/1235; A61K 39/39; A61K 2039/55572

(57) ABSTRACT

The subject of the present invention is an isolated antigen being an immunogenic form of the common enterobacterial antigen (ECA) of Gram-negative bacteria of the family Enterobacteriaceae: ECA combined with lipopolysaccharide (ECA_{LPS}); a glycoconjugate of inactivated form of this antigen with a protein, as well as compositions and vaccines containing such an antigen/glycoconjugate designed for the prevention and treatment infections caused by Gram-negative bacteria.

The subject of the present invention is an isolated antigen which is the immunogenic form of the common enterobacterial antigen of Gram-negative bacteria of the family Enterobacteriaceae, such as ECA combined with lipopolysaccharide (ECA_{LPS}), a glycoconjugate of this antigen with a protein, compositions and vaccines containing such an antigen/glycoconjugate designed for the prevention and treatment of infections caused by Gram-negative bacteria. The present invention belongs to the area of immunochemistry and immunology.

Antibacterial vaccines continue to be the least expensive and most effective method of counteracting diseases caused by Gram-negative bacteria, such as enteritis (shigellosis caused by Shigella spp., food poisoning and “typhoid fevers” caused by Salmonella spp.), plague caused by Yersinia spp., urinary tract infections caused by Escherichia coli, Klebsiella spp., Proteus spp., Serratia spp., Enterobacter, meningitis caused by Neisseria meningitidis and infections caused by enterotoxigenic (ETEC), enteropathogenic (EPEC), enterohaemorrhagic (EHEC), enteroinvasive (EIEC), enteroaggregative (EAEC), uropathogenic (UPEC), and neonatal meningitidis (MNEC) E. coli. Moreover, species of the family Enterobacteriaceae are responsible for 40-50% of hospital-acquired infections leading to septic shock and sepsis. Over half of sepsis cases in the USA are connected with bacteria of the genus Klebsiella, Escherichia, Proteus or Enterobacter, and mortality is in the range of 20-50%. Moreover, it is known that species such as Klebsiella pneumoniae, E. coli or Pseudomonas aeruginosa are drug resistance gene carriers, and the appearance of drug-resistant species in hospitals has a significant effect on the increased frequency of nosocomial (hospital-acquired) infections as well as mortality in neonates and patients undergoing complicated medical treatment. The threat of multi-drug resistant strains of Gram-negative species is extensive and on the rise from year to year, encompassing most European Union nations and the United States. For example, none of the presently available antibiotics is capable to stop the spread of drug resistance among the clinical strains of K. pneumoniae, and newly introduced antibiotics merely push back in time the need to introduce new therapeutic strategies against drug-resistant bacteria into medical practice (Woodford et al.).

Immunoprophylaxis, or protective vaccines, can greatly reduce the number of cases and complications, and even completely eradicate some diseases. Among antibacterial vaccines we differentiate cellular and acellular ones. Cellular vaccines are the oldest form. These are suspensions of chemically or thermally inactivated bacteria. Their use elicits a high degree of protection with an increased risk of side effects. Acellular vaccines, less reactive, contain purified/recombinant bacterial antigens, among which inactivated proteins are dominant, such as toxoids, i.e. diphtheria toxoid (DTd), tetanus toxoid (TTd), pertussis toxoid (PTd), or outer membrane protein. Protein antigens are T-dependent, induce a long-lasting immune response dependent from B and T lymphocyte cooperation, and thereby can be used directly as vaccine antigens.

The use of inactivated toxins: pertussis toxoid, fimbrial agglutinogens, or pertactin, is useful in the case of diseases caused by a particular Gram-negative bacteria species, such as for example Bordetella pertussis. However, this is the case for most Gram-negative bacteria, protein antigens of potential use in subunit vaccines are barely being identified using proteomics and bioinformatics using sera from covalescent patients, i.e. OMP proteins or bacterial porins (Gupta et al. 1992, Kurupati et al. 2006). Moreover, vaccines against Gram-negative bacteria based solely on antigenic proteins (inactivated toxins) often do not generate bactericidal antibodies. An example of this are the vaccine studies against Vibrio cholerae, the etiological factor of cholera. All 4 market-available vaccines contain whole bacterial cells. These are WC/rBS (killed whole cell Vibrio cholerae O1 with a purified recombinant subunit B of cholera toxin (85-90% effectiveness), Variant WC/rBS (variant of the WC/rBS vaccine not containing the recombinant B subunit of cholera toxin (66% effectiveness), CVD 103-HgR (attenuated, live genetically modified V. cholerae O1) (effectiveness 95%), and the oral vaccine Dukoral® (effectiveness 85%) (Mendoza et al. 2009). This stems from the fact that the patients' serum derived bactericidal antibodies are first and foremost directed against the surface polysaccharides of the bacteria, such as lipopolysaccharide (LPS, endotoxin) and capsular polysaccharides (capsular antigens, CPS), but not against cholera toxin. The addition to the vaccine formulation of inactivated cholera toxin did not significantly increase the effectiveness of the vaccine. Additionally, the high level of protective antibodies induced by injection of whole bacterial cells decreases rapidly, particularly in children. In the case of V. cholerae, LPS and CPS in the form of conjugates with carrier proteins seem to be the best candidates for components of acellular vaccines. It was shown that passive immunisation with antibodies against LPS of V. cholerae protected mice and rabbits against infection, and immunisation with the purified LPS of this bacteria induced protection in rabbits and humans. Detoxified LPS and O-specific polysaccharide were also used, which were conjugated with protein carriers such as cholera toxin and tetanus toxoid. These vaccines were immunogenic both in humans and animals. Studies were also conducted on the use of a synthetic O-specific polysaccharide conjugated with BSA (Grandjean et al. 2013, Kossaczka et al. 2000).

The potential non-protein vaccine antigens common to all or most Gram-negative bacteria include the aforementioned capsular polysaccharides, lipopolysaccharides and the enterobacterial common antigen (ECA). These are immunogenic surface antigens and virulence factors that induce antibodies in persons that have undergone infections by Gram-negative bacteria.

Capsular polysaccharides, also called capsular antigens or antigens K are the only saccharide antigens used in commercially available vaccines. In contrast to protein antigens, isolated and purified polysaccharides (i.e. capsular polysaccharides or LPS) are T-independent antigens, and their administration yields no long-lasting immunity. Despite being capable of activating B lymphocytes, they are not useful as vaccine antigens, because they induce no protection in children below the age of 2.

A solution to this problem is the use of polysaccharides or oligosaccharides in the form of a neoglycoconjugate, meaning the conjugates of poly- or oligosaccharides with an immunogenic protein (carrier protein) which makes this combination a desirable T-dependent antigen (Weintraub 2003).

There are commercially available conjugate vaccines that protect against infections induced by Neisseria meningitidis (with the exception of serogroup B) and Haemophilus influenzae type b (Hib). Hib vaccine was the first conjugate vaccine introduced to the market. The protective antigen in this case is the purified capsular antigen of Hib—polyribosylribitol phosphate (PRP). PRP occurs as a conjugate with the following protein carriers: tetanus toxoid (PRP-T), pertussis toxoid (PRP-D), outer membrane protein (OMP) of the cell wall of N. meningitidis type B (PRP-OMP) or as the PRP-derived oligosaccharide conjugated with a mutant diphtheria toxin molecule CRM197 (Oligo-CRM). All of the aforementioned vaccines are characterised by a high immunogenicity in adults and older children. Examples of commercial vaccines against Hib encompass conjugate vaccines of the PRP-T type: ActHib® (Pasteur Merieux) and Hiberix® (Smith-Kline Beecham), or of the PRP-OMP type: PedvaxHlB® (MSD) and PRP-HbOC with the carrier CRM197-HibTITER® (Lederle) (McIntyre et al.)

The antigen used as a vaccine constituent to immunise against meningococcal infections is the capsular polysaccharide of Neisseria meningitidis. This antigen occurs as a conjugate with tetanus toxoid (TTd) and with pertussis toxoid (DTd). To date, three monovalent vaccines have been introduced against serogroup C of N. meningitidis and a tetravalent one against serogroups A, C, W-135 and Y. Conjugate vaccines against the serogroup C are effective in children above the age of 2 months, whereas tetravalent vaccine (introduced in the US in 2005) is recommended for children of 11 years of age and older, and adults. Example of commercial vaccines against meningococci encompass: Meningitec® (monovalent C), the conjugate with pertussis toxoid CRM197 (Wyeth-Lederle), NeisVac-C® (monovalent C), the conjugate with tetanus toxoid (Baxter), and Menjugate® (monovalent C), the conjugate with pertussis toxoid CRM197 (Chiron) (McIntyre, O'Brien et al.). As a result, bactericidal antibodies are generated and the effectiveness of these vaccines is 99-100%.

The capsular polysaccharides of serotypes 4, 6B, 29V, 14, 18C, 19F and 23F of the Gram-positive bacteria Streptococcus pneumoniae in the form of conjugates with protein carrier CRM197 are constituents of a commercial vaccine against pneumococci—Prevenar® (PCV7). Kids vaccinated in the USA from year 2000 with PCV7 vaccines exhibited a drop in the incidence of infections caused by vaccine serotypes and a drop in the occurrence of carriers of such serotypes. The phenomenon of “herd immunity” (community immunity) was also observed, which was demonstrated by a drop in the rate of transmission of such strains to non-vaccinated persons, among whom significant drop in the rate of pneumococcal infections was observed. There is also a 23-valent polysaccharide vaccine (PPV23—Pneumococcal Polysaccharide Vaccine—Pneumo 23® and Pneumovax 23®), which, in children below the age of 5, has a supplementary effect following the administration of PCV7 vaccines (McIntyre, O'Brien et al.).

The examples described above show that a universal vaccine against a species of bacteria should contain such a set of antigens that is able to induce protection against the widest possible group of pathogenic strains (about 80%-90%). A universal vaccine in relation to a group of pathogens that cause nosocomial infections should induce protection against the broadest possible number of bacterial species. The aforementioned examples describe bacterial species among which the serotypes variety of the capsular antigen of human pathogenic strains is limited: 5 serotypes of Neisseria meningitidis and 7-23 dominant serotypes of Streptococcus pneumoniae. Moreover, the examples given are related to diseases caused by a single species of bacteria and only to one species in the family Neisseriaceae (N. meningitidis) and one species in Pasteurellaceae (H. influenza). At present, there are no commercial vaccines based on non-protein antigens against other species from this family, such as Shigella spp., Salmonella spp., E. coli, Pseudomonas spp., Yersinia spp., Klebsiella spp., Serratia spp., Enterobacter spp. This situation is more complicated in the case of nosocomial infections caused by Gram-negative bacteria, which leads to sepsis and septic shock. This type of infections are characterised foremost by the being mixed infections and in 40-50% of cases are caused by Klebsiella, E. coli, Enterobacter, Proteus, or Serratia. Therefore, a hypothetical vaccine composition preventing such infections would have to be characterised by a broad spectrum of antigens or be based on a single antigen common to Enterobacteriaceae.

In these cases the only solution is to prepare a polyvalent vaccine or to search for an antigen common to one or several dozen species. Effective polyvalent vaccines have been proposed that are based on 6 and 24 serotypes of capsular polysaccharides of K. pneumoniae (Campbell et al. 1996). However, the drawback of polyvalent vaccines is their complexity, which results in the high costs of preparation of each antigen, dosage optimization, as well as the selection of time-variable capsule types from pathogenic strains population. Such variability can occur as a result of evolutionary pressure caused by the vaccines used or by way of the genetically mandated alteration of the capsular antigen, the so-called “capsule switching”, observed for example for N. meningitidis (Swartley et al. 1997) or Streptococcus pneumoniae (Wyres et al. 2013). In the case of extensive capsular antigen variability in pathogenic strains, the production of a new vaccine with similar effectiveness may be technically impossible.

For this reason, there is still an extant need to identify and produce new antigens, a common one for one or a greater number of Gram-negative bacteria species, in particular species that cause nosocomial infections and infections, such those caused by Shigella spp., Salmonella spp., Klebsiella spp., Serratia spp., Enterobacter and E. coli (ETEC, EPEC, EHEC, EIEC, EAEC, UPEC, MNEC).

As mentioned above, potential non-protein antigens occurring in most or all Gram-negative bacteria, aside from capsular polysaccharides, are lipopolysaccharides (LPS) and lipooligosaccharides (LOS), and ECA.

Lipopolysaccharides and lipooligosaccharides, composed of a polysaccharide and a lipid A part, are amphiphilic molecules located on the Gram-negative bacterial cell surface (Rietschel et al. 1996). LPS protects microorganisms against the defensive mechanisms of the infected host, bile acid and hydrophobic antibiotics. It plays a significant role as a virulence factor of Gram-negative bacteria in sepsis and septic shock. Regardless of the source of LPS, this molecule, isolated from the smooth forms of bacteria, is characterised by a general structural layout encompassing three regions: (i) an O-specific polysaccharide—a polymer of repeating oligosaccharide units, characterised by a high structural variability and a determinant of the serological specificity of LPS (O-antigen specificity), (ii) core oligosaccharide—a region of limited variability within a species, (iii) lipid A—a region anchoring LPS/LOS in the membrane of the outer cell wall of Gram-negative bacteria, and composed in most Enterobacteriaceae of the disaccharide β-D-GlcpN-(1->6)-α-D-GlcpN substituted with fatty acids, phosphate groups and saccharide or non-saccharide substituents. Lipid A is linked with a core oligosaccharide by a ketosidic linkage between the Kdo residue of the core oligosaccharide and D-GlcN at the non-reducing end of the carbohydrate backbone of the lipid A. The biological activities of lipopolysaccharide are strictly connected with the structural characteristics of lipid A, which constitutes the toxic centre of LPS. The lipid A region of LPS is recognized by the elements of the innate immune mechanisms, including the CD14/TLR4/MD2 receptor complex (Lukasiewicz and Lugowski 2003).

Due to the fact that LPS is an integral component of the cell envelope of Gram-negative bacteria and induces the production of specific bactericidal antibodies as a result of immunisation with whole bacteria (for example LOS/LPS-based vaccines against V. cholerae and B. pertussis) (Grandjean, Wade et al. 2013, Kossaczka, Shiloach et al. 2000, Niedziela et al. 2005), this seems to be the ideal vaccine antigen against infections caused by Gram-negative bacteria. However, its biological activity and the fact that it constitutes the main virulence factor excludes the use of LPS/LOS as vaccine antigens in their native form. The essential conditions that must be met when using this form of the antigen in vaccines are: (i) LPS/LOS must be devoid of toxic activity (lipid A) while maintaining the structure of the immunogenic epitopes, capable of inducing antibody characterised by bactericidal and anti-endotoxin activity, and (ii) a certainty that such a modified LPS/LOS became T-dependent antigens, usually through conjugation with a protein. It is additionally desirable that the induced antibodies neutralize the undesirable biological activity of lipopolysaccharide—the molecule responsible for the development of sepsis and septic shock.

The state of the art contains many examples of using inactivated forms of LOS and LPS as vaccine antigens. It is known that specific antibodies directed against LPS of particular bacterial strains exhibit protective activities in a homologous system against strains used for immunisation.

However, the most immunogenic molecules on the surface of pathogens (LPS, CPS) also exhibit a high structural heterogeneity (serotypes O, K and H). Lipopolysaccharides are highly variable in terms of saccharide structure, particularly within the O-specific polysaccharide region, which determines the O serotype. For example, for E. coli, 180 different O serotypes (different structures of O-specific polysaccharides) have been identified (Muller-Loennies et al. 2007), with as many as 80 capsular antigen types (Whitfield 2006). For K. pneumoniae, the number of identified O-antigen types is 12. In Salmonella enteritica 46 O serotypes were identified, wherein taking into account the number of possible modifications, they may be estimated at about 2500 serotypes (Brenner et al. 2000).

It has long been known that passive immunisation with serum directed against LPS protects mice and rabbits against toxic activity of LPS, but the observed protective activity is dependent on O serotype (Muller-Loennies 2007). The heterogeneity of O-specific chains is the reason for which the vaccines based on O-specific polysaccharides are polyvalent, thus they require the use of a set of different serotypes of O-specific chain, dominant among pathogenic strains. Example solutions from prior art are based on the use of O-specific polysaccharides as conjugates with protein carriers.

The O-specific polysaccharide of S. sonnei phase I conjugated with a protein—recombinant exotoxin A of Pseudomonas aeruginosa (rEPA), protected 72% Israeli soldiers against infections (Cohen et al. 1997). Lipopolysaccharide fragment containing a core oligosaccharide substituted with several repeating units of the O-specific polysaccharide of S. sonnei LPS conjugated with BSA or with recombinant pertussis toxoid was also used as the constituent of the vaccine. This conjugate induced a much higher level of antibodies than a conjugate containing natural length O-specific polysaccharides (Robbins et al. 2009). Similar conjugates were prepared using fragments of the O-specific polysaccharide of Shigella dysenteriae type 1 and Shigella flexneri 2a (Phalipon et al. 2009, Pozsgay et al. 2007).

The variety of O-serotypes and the dynamics of sepsis and septic shock development eliminate the use of this type of antigens in preventing of nosocomial infections and septic shock. For this reason, therapeutic solutions based on LPS have been sought for many years, but ones independent of O serotype, namely ones using more conservative regions of LPS: core oligosaccharide and lipid A. Anti-lipid A antibodies have failed the hopes set for them, since the epitopes recognized by these antibodies are masked by the core oligosaccharide and O-specific chain. Clinical trials using antibodies against lipid A and core oligosaccharide type Re (incomplete deep-rough type of the core) have also failed (Muller-Loennies, Brade et al. 2007).

As a result of this, the next justified approach in vaccines against infections caused by Gram-negative bacteria was the use of core oligosaccharides. For example, 5 types of core oligosaccharides have been identified for E. coli (R1, R2, R3, R4 and K12, for which 4 dominant glycoforms were described) (Muller-Loennies, Brade et al. 2007). For the lipopolysaccharide of Salmonella, 2 types of core oligosaccharides were identified (Muller-Loennies, Brade et al. 2007, Olsthoorn et al. 1998). Two types of core oligosaccharides have been identified so far in K. pneumoniae (Muller-Loennies, Brade et al. 2007).

The example solutions encompassed by the prior art, which pertain to such non-toxic fragments of LPS or LOS are based on the use as an antigen of: (i) complete core oligosaccharides not substituted with O-specific polysaccharides or (ii) inner core oligosaccharides of LPS. In most cases, these antigens occur in purified form as conjugates with a protein carrier or desirable antibodies are generated against rough mutants of bacteria, that express on their surface core oligosaccharides not substituted with O-specific polysaccharides.

To obtain cross-reacting antibodies directed against LPS core oligosaccharides, Di Padova et al. (Di Padova et al. 1993) used rough mutants of E. coli for immunization, which represented all types of core oligosaccharides (R1, R2, R3, R4 and K12). The resulting mouse monoclonal antibodies (mAb) exhibited cross-reactivity with lipopolysaccharides isolated from about 100 clinical isolates of smooth E. coli and S. enteritica, but did not bind lipopolysaccharides of K. pneumoniae and P. aeruginosa. One of these antibodies (WN1 222-5) differed by its high affinity for the tested antigens and neutralizing activity against LPS in vivo in an animal model of septic shock. The mAb WN1-222-5 bound isolated LPS, as well as LPS complexed with high density lipoprotein, but had poor reactivity with live bacterial cells. Cross-reactivity was only observed for E. coli O18:K1 and E. coli O111:B4 (Muller-Loennies, Brade et al. 2007). Muller-Loennies identified an epitope recognized by this antibody as the internal region of the core oligosaccharide, and more specifically the epitope encompassing the terminal Hep residue and phosphate group in the respective positions 7 and 4 of the second Hep residue in the inner core region of LPS. The antibody cross-reacted with all types of core oligosaccharides of E. coli (R1, R2, R3, R4 and K-12).

Based on the antibodies produced, Di Padova obtained a humanized mAb WN1 222-5—mAb SDZ 219-800 characterised by the same specific anti-endotoxin activity in in vivo tests on septic shock models using isolated LPS (Di Padova et al. 1994). As in the case of WN1-222-5 antibodies, the authors did not show bactericidal activity of the antibodies against a broader group of live bacteria.

Lugowski et al. have obtained a series of conjugates of whole core oligosaccharides of E. coli R1, R2, R3 and R4 lipooligosaccharides with tetanus toxoid, which generated polyclonal antibodies in rabbits that cross-reacted with lipopolysaccharides isolated from smooth E. coli strains of various O-serotypes (Lugowski et al. 1996a, Lugowski et al. 1996b). Additionally, a serum directed against the prepared conjugates exhibited neutralizing activity against the endotoxins in vitro, what was demonstrated with the use of the macrophage-like cell line J744.A1. The antibodies present in these sera recognized epitopes on the surface of whole bacterial cells, as was shown using flow cytometry (FACS). The aforementioned report lacks one piece of information, related to bactericidal and protective properties of such antibodies in in vivo models of bacterial infections and septic shock.

Document EP0941738 describes the use as the antigen the conjugate of a protein carrier with conservative region of lipooligosaccharides isolated from Neisseria meningitidis, Neisseria gonorrhoeae, H. influenzae, Haemophilus ducreyi, Helicobacter pylori, Chlamydia, Proteus mirabilis, P. aeruginosa, Moraxella catarrhalis, B. pertussis, Klebsiella spp., and V. cholerae. The antigen was the LOS fragment having the structure GlcNAc-Hep₂-PEtn-Kdo₂-Lipid A, encompassing the inner core oligosaccharide region of lipooligosaccharides of the aforementioned bacteria. Sera obtained during immunisation with such conjugates exhibited cross-reactivity with LOS of N. meningitidis, H. influenzae, N. gonorrhoeae and H. pylori, and antibacterial activity in vitro against several strains of N. meningitidis.

Prior art contains few reports on potential utility (induction of immunity in in vivo models as a result of active immunisation) of antigens based on complete or incomplete core oligosaccharides. Recently, Brade et al. have published the results of an in vivo trial of cattle immunisation with conjugates of core oligosaccharides of E. coli R1, R2, R3, R4, K-12 and J-5 mutants with haemocyanin. Despite the fact that immune sera exhibited high titres of antibodies directed against the antigen, no protective activity was shown against E. coli O157 (core R1, the strain devoid of CPS). The negative results were explained by the lack of induction of antibodies directed against the O-specific polysaccharide (Brade et al.).

The lack of anti-endotoxin activity in in vitro tests of sera produced upon immunisation with conjugates of incomplete core oligosaccharide (mutant E. coli J5) with TTd and killed cells of E. coli J5 was also shown by Lukasiewicz J. et al. (Lukasiewicz et al. 2002). Despite high antibodies titres against the antigen, they were not capable of neutralizing native endotoxins isolated from smooth strains of E. coli. The lack of desired activity was explained as resulting from the unnatural conformation and the lack of key conformational epitopes in the incomplete core oligosaccharide J5, despite the fact that these are common for many bacteria of the family Enterobacteriaceae.

The ECA molecule, the common antigen of the Enterobacteriaceae family was discovered in 1962 r. by Kunin et al. (Kunin 1963, Kunin and Beard 1963). It was identified on the surface of most species of Enterobacteriaceae, such as E. coli, Salmonella spp., Shigella spp., Klebsiella spp., Edwarsiella, Enterobacter, Citrobacter, Serratia, Proteus, Yersinia spp., Erwinia, Edwarsiella, Plesiomonas shigelloides, Aeromonas hydrophilia, or Hafnia alvei. Three forms of ECA occur in nature: ECA_{PG}, ECA_{CYC}, and ECA_{LPS}, whose common part, regardless of the species of bacteria, are poly- or oligosaccharides composed of a trisaccharide repeating unit with the following structure: [→3)-α-D-Fuc4NAc-(1→4)-β-D-ManNAcA-(1→4)-α-D-GlcNAc-(1→]ₙ, where Known modifications of this structure relate to the substitution of GlcNAc with a GlcN residue and non-stoichiometric substitution with O-acetyl groups (Dell et al. 1984, Lugowski 1987). In the case of ECA_{PG}, such poly- and oligosaccharides substitute phosphatidylglycerol (PG) (Kuhn et al. 1983, Rick et al. 1998). ECA_{CYC} is a cyclic form containing from 4 to 6 ECA trisaccharide repeating units (Dell, Oates et al. 1984, Fregolino et al., Lugowski and Romanowska 1978, Lugowski and Romanowska 1978, Romanowska et al. 1978). The ECA_{LPS} corresponds to the ECA polysaccharide substituting the core oligosaccharide region of lipopolysaccharide. ECA_{PG} and ECA_{LPS} are anchored in the outer membrane of the cell wall by phosphoglyceride and lipid A, respectively. The ECA_{CYC} lacks the phospholipid anchor, and its localization in the bacterial cell has not been determined unequivocally. Aside from the chemical differences, the three identified ECA forms differ in their immunogenicity, meaning in their ability to induce specific antibodies as a result of immunisation. The only immunogenic form, namely a molecule that is able to induce antibodies in free form or bound to the surface of a bacterium, is thought to be ECA_{LPS}. This was demonstrated a number of times by the research of Kunin et al. for the serum directed against E. coli O14:K7⁻, a classical strain thought to be one that synthesizes ECA_{LPS} (Kunin 1963, Kunin and Beard 1963). A serum containing anti-E. coli O14 antibodies cross-reacted with E. coli bacteria of other serotypes, which was indicative of the presence of an antigen on the surface common to this species of E. coli. The cross-reactive antibodies could be removed from the anti-E. coli O14 serum through absorption onto an extract obtained from any given strain of E. coli. Only antibodies solely reactive to homologous strains remained in the serum. These results showed that ECA occurs solely as a hapten (non-immunogenic, i.e ECA_{CYC} and ECA_{PG}) in most evaluated strains, whereas in E. coli O14 and several other strains, it occurs as an immunogenic form (ECA_{LPS}).

In contrast to the structurally well-known ECA_{PG} and ECA_{CYC}, extant evidence of the existence of ECA_{LPS} comes mainly from serological tests. For the first time, the presence of the ECA_{LPS} was noted in rough E. coli mutants, in which lipooligosaccharides contain complete core oligosaccharide structures of E. coli R1, R4 and K-12 (Kuhn et al. 1988) and R2 and R3 (Duda et al. 2009) type, rough mutants of Yesrsinia enterocolitica O:3 (core oligosaccharide type Ra) and in smooth strains of Y. enterocolitica O:3 Ye75S (Radziejewska-Lebrecht et al. 1998). The detection of ECA_{LPS} in most cases was based on the immunoblotting and ELISA assays using monoclonal antibodies obtained as a result of immunisation with rough strains of E. coli K-12 (strain C600) or E. coli R4 (O serotype14:K7⁻), which are both known for the fact that they express the antigen ECA_{LPS} on their surface. The presence of ECA_{LPS} was also shown indirectly using chemical methods and immunogenic (in terms of ECA) strains of E. coli (Kiss et al. 1978).

The only extant prior art solutions based on the use of ECA as an antigen designed to generate specific anti-ECA antibodies (polyclonal or monoclonal) relate to immunisation with whole cells of rough mutants, i.e. E. coli O14:K7⁻ or isolated ECA_{CYC} in the form of complexes or conjugates with proteins.

Lugowski et al. obtained a neoglycoconjugate of de-O-acetylated ECA_{CYC} isolated from S. sonnei phase II with tetanus toxoid (Lugowski et al. 1983, Lugowski and Romanowska 1991). This, in contrast to free forms of ECA_{CYC} and mixtures of ECA_{CYC} with a protein, generated high titres of specific polyclonal anti ECA antibodies. In the obtained conjugates, however, ECA_{CYC} was used, which does not generate any protective antibodies in nature. Furthermore, the authors performed no tests to show the cross-reactivity of the resulting serum with various Gram-negative bacteria species and isolated lipopolysaccharides. This report mentions no information on the topic of bactericidal and protective activity of such antibodies.

Peters H. et al. have obtained monoclonal antibody produced by a hybridoma cell line that specifically recognizes the immunogenic form of ECA—the antigen ECA_{LPS} (Peters et al. 1985). These cell lines were obtained using the prior art procedure of immunizing mice with cells of the rough mutant E. coli C600 that produces LOS with the K-12 core type and the immunogenic form of ECA—ECA_{LPS}. The resulting monoclonal antibody very poorly bound whole smooth and rough E. coli cells, which was shown using ELISA by coating the plates with whole bacterial cells. A positive reaction in ELISA assay was observed only in extracts of boiled bacteria and some isolated antigens, such as lipooligosaccharides lacking O-specific chains: LOS of E. coli O14:K7 (R4), R1, K-12 and R2. In the case of smooth LPS of Salmonella Typhimurium and E. coli, the described mAb recognized mainly high-molecular weight, non-immunogenic ECA_{PG} forms and did not bind ECA_{LPS} epitopes outside of homological systems, as was shown by immunoblotting of electrophoretically separated LOS and LPS preparation. The authors, despite their claims of broad cross-reactivity of the antibodies, did not test the cross-reactions with different species of Gram-negative bacteria, and isolated lipopolysaccharides. In the aforementioned reports, there is also a lack of information on the bactericidal activity of such antibodies. Also, immunisation with whole bacterial cells does not guarantee that the obtained mAb is directed against ECA_{LPS}.

Bottger et al. used antibodies obtained by Peters H. et al. for screening studies based on ELISA on the cross-reaction of this antibody with whole cells of 90 species of Gram-negative bacteria (Bottger et al. 1987). As they themselves stressed, because the tested monoclonal antibody was not capable of reacting with native bacteria due to the masking of the ECA_{LPS} epitopes by O-specific chains and capsular antigens, during the ELISA they used boiled bacteria extracts. This drawback of the indicated monoclonal antibody, consisting of the inability to react with native bacteria, excluded its use as a diagnostic antibody, let alone a therapeutic one. Similarly to earlier studies, the authors performed no experiments relating to bactericidal and protective properties of this antibody.

Document EP0387850 describes monoclonal antibodies produced by immortalized B lymphocytes from patients with enteric infections. These antibodies cross-reacted in an ELISA test with ECA isolated from Salmonella Montevideo. This antigen was used to select B lymphocytes. According to the cited immunoblotting results and available literature data, this antigen was likely ECA_{PG}. Using ELISA on whole bacterial cells killed with phenol, the antibody inventors showed cross-reactions with Salmonella Typhimurium, K. pneumoniae, S. marcescens, P. mirabilis, E. coli, P. aeruginosa. The antibodies exhibited protective properties in a mouse in vivo model of infection with S. marcescens. To summarise, the obtained monoclonal antibodies were an effect of selection using a non-immunogenic form of ECA—ECA_{PG}. Based on the results shown, it cannot be stated what target antigen on the surface of the bacterial cell was recognized by the monoclonal antibodies in question. Taking into account the antigen used for selection, this is likely ECA_{PG}.

WO 92/17603 discloses monoclonal antibodies obtained as a result of immunisation of mice with a formulation containing a suspension of cells of the rough mutant of E. coli O14:K7⁻ (classic strain producing ECA_{LPS}). The mAbs were produced using known procedures and selected via ELISA assay using various strains of Enterobacteriaceae. The resulting antibodies exhibited positive reactions with a large group of Gram-negative bacteria species, including E. coli, Shigella flexnerii, S. sonnei, Salmonella spp., Citrobacter freundii, K. pneumoniae, Enterobacter spp., H. alvei, Serratia spp., and Proteus spp. The authors, however, did not give any criteria for evaluating the mAb-bacteria reaction as positive or negative (antibody titre). Similar to earlier efforts, the authors did not perform bactericidal activity assays and investigate protective properties of this antibody. Additionally, according to the authors of the patent application PCT9217603, this document supposedly disclosed an epitope recognized by these antibodies indicating the immunogenic form of ECA_{LPS}. This disclosure, however, is not supported by any results backing up such a conclusion, including the identification, isolation and structural analysis of a real antigen. Most of all it lacks a positive result of the interaction between this mAb and the isolated and purified antigen.

The aforementioned report by Kiss et al. (Kiss, Rinno et al. 1978) describes an attempt to isolate ECA_{LPS} and to show a covalent bond between ECA and LOS in the E. coli O8⁻:K27^{-and} O14:K7⁻ mutants with a mutation in the rfb and rfaL genes responsible for LPS biosynthesis. The bacteria were cultured in a medium supplemented with [¹⁴C]-D-GlcNAc in order to isotopically label D-GlcNAc and D-ManNAcA present in the trisaccharide repeating unit of ECA. LOS was isolated via PCP (phenol/chloroform/petroleum ether extraction) and subjected to mild hydrolysis with an acetic acid solution, and the resulting poly- and oligosaccharides were fractionated on DEAE-cellulose. The authors obtained fractions with increased radioactivity and containing ECA trisaccharide components. The publication shows no evidence of the covalent bond between ECA and the core oligosaccharide. The final conclusion is that in the case of type R1 and R4 oligosaccharides, ECA substitutes a region common for both cores, which is false in light of the results disclosed in the present invention (Gozdziewicz T. K. et al, 2014).

Lugowski C. (Post. Hig. Med. Dośw., 1987) attempted to isolate ECA_{LPS} from S. sonnei phase II LPS via PCP extraction. The purified LPS was hydrolysed with an acetic acid solution, and the released poly- and oligosaccharides were fractionated on a column with Bio-Gel P-4. The fraction containing the antigen recognized by anti-ECA_{CYC}-TT antibodies was rechromatographed on Bio-Gel P-10. The resulting fractions were subjected to 1D ¹H NMR analysis indicating that the obtained fractions contain components characteristic for the core oligosaccharides of S. sonnei phase II LOS and ECA trisaccharide repeating unit. This report, however, presented no proof of the covalent bond between ECA and the core oligosaccharide.

Thus, despite much evidence of the occurrence of ECA_{LPS} in Gram-negative bacteria, solely based on serological tests, for several decades it has been impossible to isolate, purify and analyze the structure of the ECA_{LPS} antigen present on the surface of any bacteria of the family Enterobacteriaceae, and by the same token to obtain an antigen for generating antibodies with a broad spectrum of activity against Gram-negative bacteria (directed against ECA and core region of LPS).

The prior art problem is to deliver a universal vaccine directed against a broader group of Gram-negative bacteria. To date such vaccine has not been commercialised. There is thus a need for obtaining vaccines with a minimal number of antigens ensuring protection against a broad group of heterologous strains within a species, as well as against different species of Gram-negative bacteria.

The goal of the present invention is to deliver an isolated antigen for use in vaccines protecting against infections caused by Gram-negative bacteria, having the following characteristics: (i) the generation of antibodies with a broad cross-reactivity, (ii) the generation of bactericidal antibodies, capable of binding epitopes on the surface of live bacterial cells, and (iii) the generation of antibodies with anti-endotoxin activity.

The goal of the present invention is to deliver an isolated antigen, common for one or more species of Gram-negative bacteria, in particular species causing nosocomial infections and infections such as caused by Shigella spp., Salmonella spp., Klebsiella spp., Serratia spp., Enterobacter and E. coli (ETEC, EPEC, EHEC, EIEC, EAEC, UPEC, MNEC).

These goals have been achieved in the present invention.

The subject of the present invention is isolated immunogenic antigen with the following structure:
[ECA repeating unit]ₙ-core oligosaccharide-lipid A,
   where the repeating biological ECA repeating unit has the structure: [→3)-α-D-Fuc4NAc-(1→4)-β-D-ManNAcA-(1→4)-α-D-GlcNAc-(1→]ₙ, a n≧1, and where core oligosaccharide and lipid A are from strains of Gram-negative bacteria, wherein lipid A is in detoxified form.

Preferably, ECA repeating unit is connected by a glycosidic bond with a core oligosaccharide of Gram-negative bacteria.

Preferably, the core oligosaccharide originates from the lipopolysaccharide or lipooligosaccharides of E. coli, Salmonella, Shigella, Klebsiella, Edwarsiella, Enterobacter, Citrobacter, Serratia, Proteus, Yersinia, Erwinia, Plesiomonas, Aeromonas.

More preferably, the core oligosaccharide originates from lipooligosaccharides of S. sonnei phase II.

The next subject of the present invention is an isolated immunogenic antigen, characterised in that it originates from Shigella sonnei phase II and has the structure [ECA repeating unit]ₙ-core oligosaccharide (dECA_{LPS}) with the formula (1):

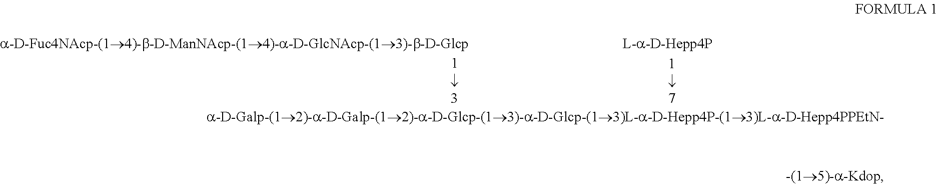

The next subjects of the present invention are pharmaceutical compositions containing an isolated antigen defined above and a pharmaceutically permissible carrier.

The next subjects of the present invention are pharmaceutical compositions containing an isolated antigen defined above and a pharmaceutically permissible carrier for the treatment of diseases caused by Gram-negative bacteria, particularly of the family Enterobacteriaceae.

Preferably, the disease is selected from a group consisting of nosocomial infections and septic shock and enteritis.

Preferably, the disease is caused by infections by bacteria selected from a group consisting of Shigella spp., Salmonella spp., Klebsiella spp., Serratia spp., Enterobacter and E. coli (ETEC, EPEC, EHEC, EIEC, EAEC, UPEC, MNEC).

The next subject of the present invention is the use of pharmaceutical compositions containing an isolated antigen as defined above and a pharmaceutically permissible carrier in the manufacturing of a preparation for the treatment of diseases caused by Gram-negative bacteria, particularly bacteria of the family Enterobacteriaceae, selected from a group encompassing nosocomial infections, septic shock, and enteritis.

The next subjects of the present invention are antibodies that bind to epitopes of the antigen defined above that cross-react with strains of Gram-negative bacteria, particularly of the family Enterobacteriaceae.

Preferably, the cross-reaction occurs with strains of bacteria selected from a group encompassing S. sonnei phase H, S. enteritica, E. coli (O39, O100, O18, O6), H. alvei and z ECA_{PG} S. sonnei phase II, S. enteritica, E. coli, Proteus vulgaris, P. mirabilis, K pneumoniae, P. shigelloides, Citrobacter.

The next subject of the present invention is a glycoconjugate containing an isolated immunogenic antigen defined above and a carrier protein.

Preferably, the pharmaceutically permissible carrier is selected from a group encompassing tetanus toxin or toxoid (TT/TTd), diphtheria toxin or toxoid (DT/DTd), diphtheria toxin mutant CRM197, exotoxin A Pseudomonas, toxin B or toxoid of Clostridium difficile, cholera toxin or toxoid (TC/TCd), toxins of Streptococcus group A Streptococcus pneumoneae pneumolysin, filamentous haemagglutinin (FHA) of Bordetella pertussis and fragments thereof, Neisseria gonorrhoeae pilum and pilin, outer membrane proteins (OMP).

The next subjects of the present invention are vaccines containing the glycoconjugate defined above and a pharmaceutically permissible carrier and optionally an adjuvant.

The next subject of the present invention is the use of vaccines containing the glycoconjugate defined above, a pharmaceutically permissible carrier and optionally an adjuvant in the manufacturing of a preparation for the prevention and treatment of diseases caused by Gram-negative bacteria, particularly of the family Enterobacteriaceae, selected from a group encompassing nosocomial infections and septic shock and enteritis.

Preferably, the disease is caused by infections of bacteria selected from a group Shigella spp., Salmonella spp., Klebsiella spp., Serratia spp., Enterobacter and E. coli (ETEC, EPEC, EHEC, EIEC, EAEC, UPEC, MNEC).

The next subject of the present invention is a method of obtaining the isolated antigen defined above from Gram-negative bacteria encompassing the following stages:
(i) isolation of LOS or LPS from the cell wall of Gram-negative bacteria,
(ii) detoxification of LOS or LPS,
(iii) fractionation of poly- and oligosaccharides using gel chromatography and/or HPLC-MS on Zic-HILIC®, in order to separate dECA_{LPS} from ECA_{PG} and ECA_{CYC},
(iv) identification of fractions containing dECA_{LPS} based on the obtained mass spectra.

Preferably, isolation of LOS or LPS is conducted using water-phenol extraction or PCP. Preferably, detoxification occurs by acid hydrolysis, and then the removal of lipid A by centrifugation.

The present application discloses the antigen ECA_{LPS} with the formula 1 (FIG. 4) isolated from S. sonnei phase II with a fully defined structure (Example 2 and 3) and other ECA_{LPS} antigens derived from strains and species of bacteria of the family Enterobacteriaceae that can be easily identified and isolated using the method disclosed in the present patent application (Example 1). The authors of the present invention have disclosed for the first time the complete structure of the immunogenic form of ECA—ECA_{LPS} isolated from S. sonnei phase II, structure of biological repeating unit of ECA_{LPS}. We also disclosed a position where the core oligosaccharides is substituted with the ECA polysaccharide.

The structure of ECA_{LPS} is shown by the formula: [ECA repeating unit]ₙ-core oligosaccharide-lipid A, where the structure of the region [ECA repeating unit]ₙ-core oligosaccharide (dECA_{LPS}) characteristic for dECA_{LPS} S. sonnei phase II is shown in the FIG. 4 (Gozdziewicz T. K. et al., 2014).

Furthermore the present invention discloses methods of identifying and isolating ECA_{LPS} from bacteria for use in obtaining vaccine antigens ECA_{LPS} from various species of Gram-negative bacteria. The method of isolating dECA_{LPS} is described in Example 1 and it encompasses 4 stages: (i) isolation of LOS or LPS (ii) detoxification LOS or LPS, (iii) fractionation of poly- and oligosaccharides using gel chromatography and/or HPLC-MS on Zic-HILIC®, in order to separate dECA_{LPS} from ECA_{PG} and ECA_{CYC} (iv) identification of dECA_{LPS} based on the obtained mass spectra.

The disclosed method makes it possible to isolate ECA_{LPS} from LPS preparation of smooth and rough strains of Gram-negative bacteria, universal for bacteria of the family Enterobacteriaceae. The isolation of LPS is performed using prior art methods, which encompass, but not limited to, phenol-water extraction and PCP extraction. The detoxification of ECA_{LPS} in order to obtain dECA_{LPS} is performed using standard methods, such as the mild acid hydrolysis of LPS/LOS or de-O-acylation LPS/LOS using NaOH or hydrazinolysis, than the water-insoluble fraction of lipid A or fatty acids is removed by centrifugation. Alternatively, such a dECA_{LPS} may be isolated from bacterial mutants with mutation of genes responsible for the biosynthesis of ester-linked lipid A fatty acids.

The fractionation of poly- and oligosaccharides in order to isolate dECA_{LPS} can be performed using known prior art chromatographic methods, including, but not limited to gel chromatography, ion exchange chromatography, affinity chromatography, chromatography using the Zic-HILIC® gel is particularly preferable. The resulting dECA_{LPS} is free of contaminants, such as ECA_{PG} and ECA_{CYC}. The antigen dECA_{LPS} is identified based on the obtained mass spectra ESI-MSⁿ and/or MALDI-TOF and optionally NMR spectra (Example 2). It is assumed that the disclosed antigen can undergo modifications identified for ECA_{PG} and ECA_{CYC}, such as O-acetylation and/or substitution of D-GlcNAc in the ECA trisaccharide by a D-GlcN residue (Dell, Oates et al. 1984, Lugowski 1987, Lugowski and Romanowska 1978, Romanowska, Katzenellenbogen et al. 1978).

It is also disclosed that conjugates of the detoxified form of ECA_{LPS} with tetanus toxoid (Example 3), used in the immunisation or rabbits, induced the production of an immune serum directed against ECA_{LPS}. Immunisation with the aforementioned glycoconjugate (Example 4) induces the production of anti-ECA_{LPS} polyclonal antibodies in an individual that recognize all forms of ECA present on the surface of Gram-negative bacteria: ECA_{LPS}, ECA_{PG} and ECA_{CYC}, as shown in a dot-blot test, immunoblotting and ELISA (Example 5) using purified ECA_{CYC} and preparations of S. sonnei phase II containing LOS, ECA_{LPS} and ECA_{PG}. The produced antibodies cross-reacted with various preparations of LPS/LOS and native Gram-negative bacteria (Example 6). Therefore, such an antigen and glycoconjugate are of potential use as components of vaccines for the prevention and treatment of infections caused by Gram-negative bacteria, in particular in the prevention of nosocomial infections.

W disclose an example (Example 3) of the conjugation of the dECA_{LPS} antigen with a protein based on the use of the aldehyde group produced in dECA_{LPS} and the amine group of the protein carrier which is conducted using known prior art reductive amination reactions. Methods of conjugating proteins with poly- and oligosaccharides are known in the state of the art and have been described by Hermanson G. T in the book “Bioconjugation techniques” (Hermanson 2008).

The present invention also relates to vaccines containing such an antigen and/or its glycoconjugate for the treatment of infections caused by Gram-negative bacteria, in particular bacteria of the family Enterobacteriaceae.

The present application discloses the ECA_{LPS} antigen isolated from S. sonnei phase II with a fully defined structure and other ECA_{LPS} antigens derived from strains and species of bacteria of the family Enterobacteriaceae, which may easily be isolated using the method disclosed in the present patent application and analysed using methods commonly known to a specialist in the art. Immunisation with the aforementioned antigen induces in an individual bactericidal polyclonal antibodies cross-reactive with all forms of ECA and various species of Gram-negative bacteria.

Taking into account present prior art relating to vaccines against infections caused by Gram-negative bacteria, and in particular vaccines based on the antigen common to many strains of a given species or many species of Gram-negative bacteria (core oligosaccharides of LOS/LPS, the inner core oligosaccharides of LOS/LPS), in particular bacteria of the family Enterobacteriaceae, the present invention relates to a novel antigen common to most bacteria of the family Enterobacteriaceae: ECA_{LPS}. The present document discloses for the first time the complete structure of this antigen.

Regardless of species, the ECA_{LPS} antigen retains an identical structure in the ECA repeating units region. The ECA polysaccharide region is not subject to the variability described for capsular polysaccharides or the O-specific polysaccharides, and thereby vaccines based thereon, i.e. vaccines against nosocomial infections, do not need to contain many antigens and may be combined with other commercially available vaccines forming combined vaccines against several categories of microorganisms (bacteria, viruses). The disclosed antigen and its conjugates makes it possible to greatly reduce the quantity of antigens needed to make a polyvalent vaccine directed against the dominant pathogens in the group of Gram-negative bacteria, in particular of the family Enterobacteriaceae.

In contrast to solutions using ECA_{CYC} as the antigen, the advantage of the present invention is based on the use of the immunogenic form of ECA—detoxified ECA_{LPS} combined with the core oligosaccharide. As the sole natural form of ECA it induces specific antibodies in an individual subjected to immunisation or following infection. The use of complete core regions as well as the fact that they occur in a from substituted with ECA is a guarantee of the retaining of the natural conformation of core oligosaccharide region, present in the smooth forms of lipopolysaccharide. The antibodies generated as a result of immunisation with the disclosed conjugates recognize the antigen well exposed and bound to the cell wall of bacteria and exhibit bactericidal properties against rough mutants of E. coli lacking CPS, rough mutants of E. coli that produce CPS, as well as a number of smooth strains of E. coli that may or may not produce the capsular antigen, including E. coli O104 (enteroaggregative strain, EAEC). Moreover, in immunoblotting using anti-dECA_{LPS} immune sera we observed cross-reactions with ECA_{LPS} of S. sonnei phase II, S. enteritica, E. coli (O39, O100, O18, O6), H. alvei and with ECA_{PG} of S. sonnei phase II, S. enteritica, E. coli, Proteus vulgaris, P. mirabilis, K. pneumoniae, P. shigelloides, Citrobacter. The disclosed antigen generates antibodies, which are free of the problem of impeded access to epitopes on the surface of living bacteria with smooth LPS, which often occurs in antibodies directed against the incomplete, deep regions of LPS core oligosaccharide.

In addition, the present invention delivers an antigen that induces antibodies directed against all forms of ECA (ECA_{PG}, ECA_{LPS} and ECA_{CYC}) and, dependent of the core oligosaccharide used, also against epitopes within core oligosaccharide region. In the case of immunisation with conjugates containing the dECA_{LPS} form of S. sonnei phase II or E. coli R1, the generated antibodies exhibit cross-reactions with ECA_{PG}, ECA_{LPS} and ECA_{CYC} and lipopolysaccharides with the core types R1, R2, R4 and K-12 (E. coli, S. sonnei) and Ra (Salmonella) (Lugowski, Jachymek et al. 1996a, Lugowski, Niedziela et al. 1996b). A specialist in the art will also see the advantage of the presence in said antigen of epitopes of the core regions of LPS/LOS. Studies of Lugowski et al. (Lugowski, Jachymek et al. 1996a, Lugowski, Niedziela et al. 1996b) have shown the high potential of complete core oligosaccharides of type R1 and R3 in the generation of antibodies with cross-reactivities against R2 and R3 and R2 and Ra core types, respectively. The example disclosed in the present invention of a single dECA_{LPS} antigen with the core type R1 concentrates 6 different epitopes (R1, R2, R3, ECA_{LPS}, ECA_{PG}, ECA_{CYC}).

The advantage of the disclosed antigen/conjugates over extant prior art antigen/conjugates is based on the prevalence of its epitopes among bacteria of the family Enterobacteriaceae and the exposure of these epitopes in a naturally occurring form on the surface of Gram-negative bacteria. The natural combination of ECA with the core oligosaccharide of LPS broadens the scope of the proposed epitopes, thereby making it possible to decrease the number of essential antigens in universal vaccines directed against Gram-negative bacteria, in particular bacteria of the family Enterobacteriaceae.

The active immunisation form proposed by the authors of the present invention answers the problem of dynamics of sepsis and septic shock, which prevent the identification of the serotype of the pathogen due to the lack of rapid diagnostic methods and therefore the use of specific monoclonal antibodies. A significant element in the prevention of septic shock is not only the inactivation of the released endotoxin from deceased bacteria (antibiotic activity), but first and foremost killing the invading pathogen (bactericidal effect). The monoclonal antibodies described previously are directed against the deep, incomplete core oligosaccharide regions, and exhibited a promising broad cross-reactivity in in vivo sepsis models using isolated LPS, but very limited against live bacteria. The advantage of active immunisation (protective vaccination) over passive (administration of monoclonal antibodies) in the prevention of sepsis and the development of septic shock is also based on the generation of immune memory and antibodies with a much longer half-life as compared to monoclonal antibodies, including humanized monoclonal antibodies.

Vaccines are at present the sole alternative to antibiotics and the only effective method of preventing infections. This is significant in light of increasing drug resistance, particularly in species causing nosocomial infections.

Taking into account the broad specificity of antibodies directed against the conjugates of dECA_{LPS}-TTd and their bactericidal activity, this conjugate is potentially useful as a component of vaccine against infections caused by Gram-negative bacteria, in particular for the treatment of both infections caused by a single species of bacteria (enteritis, urinary tract infections), as well as for the treatment of mixed infections (nosocomial infections, sepsis). In particular, the present invention can be of use in preventing and/or treating infections caused by Gram-negative bacteria, in particular bacteria of the family Enterobacteriaceae, such as Shigella spp., Salmonella spp., Klebsiella spp., Serratia spp., Proteus, Enterobacter, Plesiomonas and E. coli (ETEC, EPEC, EHEC, EIEC, EAEC, UPEC, MNEC).

In particular, the disclosed antigen and its conjugates are of potential use as components of vaccine against nosocomial infections, in which 40-50% of etiological factors are bacteria of the genera Proteus, Klebsiella, Enterobacter and Escherichia. The disclosed antigen and its conjugates might be used for active immunisation of individuals at increased risk of sepsis (patients undergoing complex therapies, immunosuppressed, AIDS patients with recurrent infections caused by bacteria of family Enterobacteriaceae).

### FIGURE DESCRIPTIONS

FIG. 1 Mass spectra of the analysed fractions of dECA_{LPS}. 1a. ESI MS spectrum in the negative ion mode of high-molecular fractions of dECA_{LPS} containing the core oligosaccharide of S. sonnei phase II combined with the 2, 3 and 4 repeating units of the ECA chain. 1b. ESI MS spectrum in the positive ion mode of dECA_{LPS} fractions containing a single ECA repeating unit. 1c. MS/MS spectrum in the positive ion mode of the ion at m/z 839.21 corresponding to one of the dECA_{LPS} glycoforms with the inset.

FIG. 2 Anti-dECA_{LPS} serum reactions with selected lipopolysaccharides. 2a. Gel electrophoresis separation in a 15% acrylamide gel, followed by silver staining of lipopolysaccharides. 2b. Immunoblotting of lipopolysaccharides with 1200-fold diluted anti-dECA_{LPS}

FIG. 3 ELISA of reactions of anti-dECA_{LPS} serum (dilutions: 1/80-1/20480) with living cells of selected Gram-negative bacteria. A₄₀₅—absorbance at 405 nm. The results are presented as an average of two replicates.

FIG. 4 Structure of dECA_{LPS} isolated from S. sonnei phase II.

FIG. 5 ¹H and ¹³C NMR chemical shifts of dECA_{LPS} isolated from S. sonnei phase II (FORMULA 1, FIG. 4).

FIG. 6 Selected inter-residue NOE and ³J_{H,C} connectivities from the anomeric atoms of dECA_{LPS} dodecasaccharide isolated from S. sonnei phase II LOS. Data indicating the covalent linkage between ECA and LOS are shown in bold.

FIG. 7 The bactericidal titers of the anti-dECA_{LPS}-TTd sera against selected strains of Gram-negative bacteria.

The invention is illustrated by the following example embodiments

### EXAMPLE 1

### The Isolation of ECA_{LPS}

A rough strain of the Gram-negative bacterium Shigella sonnei phase II was obtained from the Polish Microorganism Collection. The bacteria were cultured on agar plates for 24 hours in 37° C., suspended in PBS and the prepared inoculum was used for a culture in a 9 L fermenter for 12-18 hours in liquid LB medium or Davis mineral medium supplemented with glucose, casein hydrolysate and yeast extract. After the culture, the bacteria were killed with 0.5% phenol, centrifuged and lyophilised. ECA_{LPS} was isolated according to a procedure encompassing these 4 stages:

1) Isolation of LOS or LPS Using Water-Phenol Extraction or PCP.

Lipooligosaccharides were isolated from a bacterial mass using phenol-water extraction according to Westphal. The aqueous phase was dialysed against water for 5 days, filtrated and lyophilised. Lipooligosaccharides were purified via triple ultracentrifugation at 100 000×g for 6 h.

2) Detoxification of LOS or LPS as a Result of Acid Hydrolysis and Removal of Lipid A by Centrifugation,

The purified lipooligosaccharide fraction was detoxified via hydrolysis of the acid labile bond between the core oligosaccharide Kdo residue and lipid A in 1.5% acetic acid at 100° C. for 30 min. The mixture was centrifuged (40 000×g, 20 min.) and the supernatant containing the oligosaccharides was collected, wherein the precipitated lipid A is separated as a pellet. The collected supernatant was lyophilised.

3) Fractionation of Poly- and Oligosaccharides Using Chromatographic Methods,

The obtained oligosaccharides were separated using gel chromatography on a column with BioGel P-10 (1.8×90 cm) equilibrated with 0.02M pyridine buffer (pH 5.4). The eluted fractions were monitored by a Knauer differential refractometer and collected for further analyses. h. ECA-containing fractions were identified using dot-blotting and anti-ECA_{CYC} antibodies and/or antibodies against anti-ECA_{LPS}. Fractions containing epitopes characteristic of ECA_{LPS} were collected and lyophilised. When it was necessary to differentiate and/or purify the ECA_{LPS} from ECA_{PG} and ECA_{CYC} we used HPLC-MS with Zic-HILIC® column.

4) Identification of dECA_{LPS} Based on the Obtained Mass Spectra.

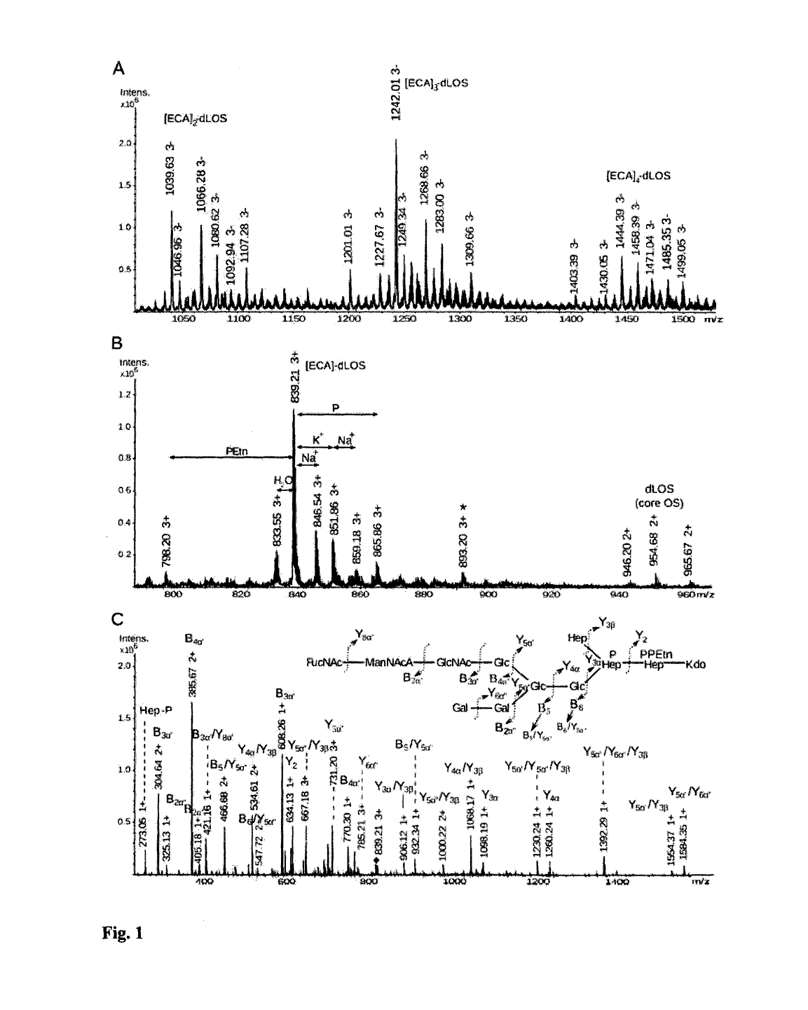

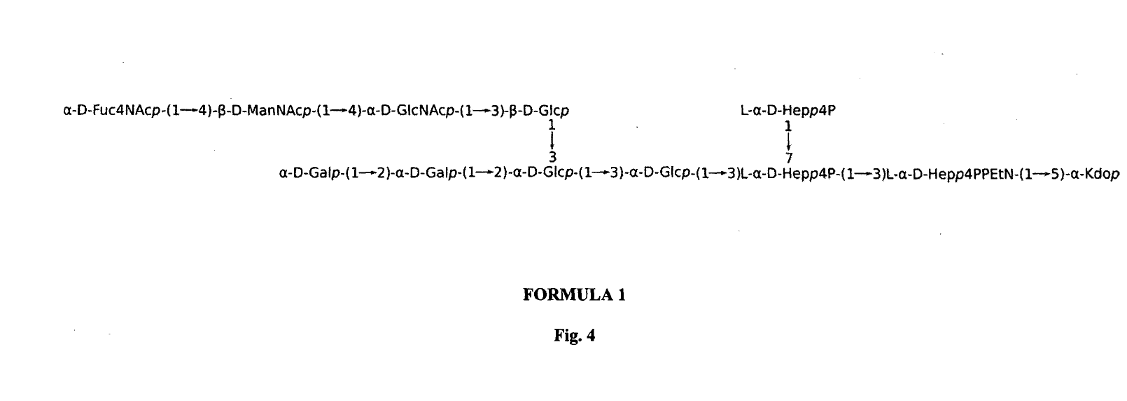

The fractions were analysed using mass spectrometry. Samples of oligosaccharides were dissolved in a mixture of acetonitrile/water/formic acid (50:50:0.5), 50 μg/ml. Measurements were made on amaZon SL (Bruker Daltonik) spectrometer equipped with an ion trap and an Apollo II electrospray ioniser (ESI). The spectra were recorded in negative and positive ion modes. In the mass spectra of high-molecular fractions ions corresponding to core oligosaccharides combined with two ([ECA]₂-OS), three ([ECA]₃-OS) and four ([ECA]₄-OS) ECA repeating units respectively were identified (FIG. 1a). Analysis of the low-molecular weight fractions showed the presence of ions corresponding to the core oligosaccharide substituted with one ECA repeating unit, ECA-OS (FIG. 1b). Fragmentation spectrum of the ion representing [ECA]₁-OS (FIG. 1c) was obtained. The analysis performed made it possible to conclude that the ECA trisaccharide is bound to the core oligosaccharide in the so-called hexose region, with the highest probability of the terminal glucose of the core oligosaccharide as a place of substitution (FORMULA 1, FIG. 4) (Gozdziewicz T. K. et al., 2014).

### EXAMPLE 2

### Structural Analysis of dECA_{LPS}

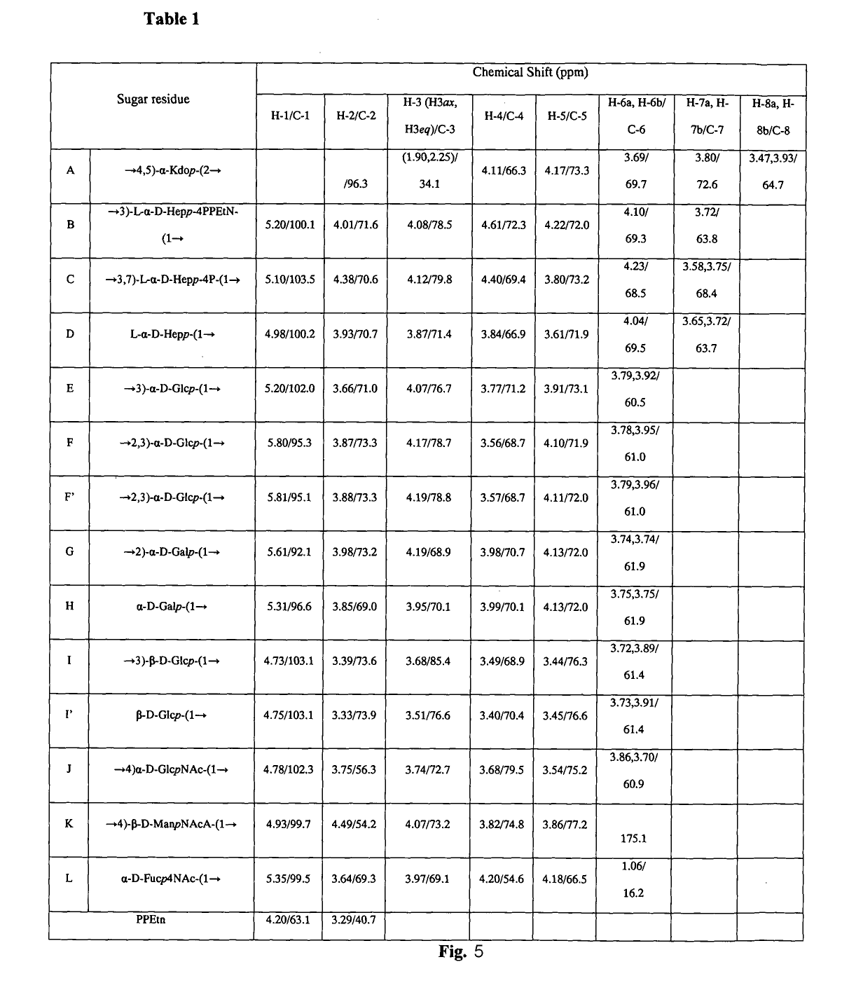

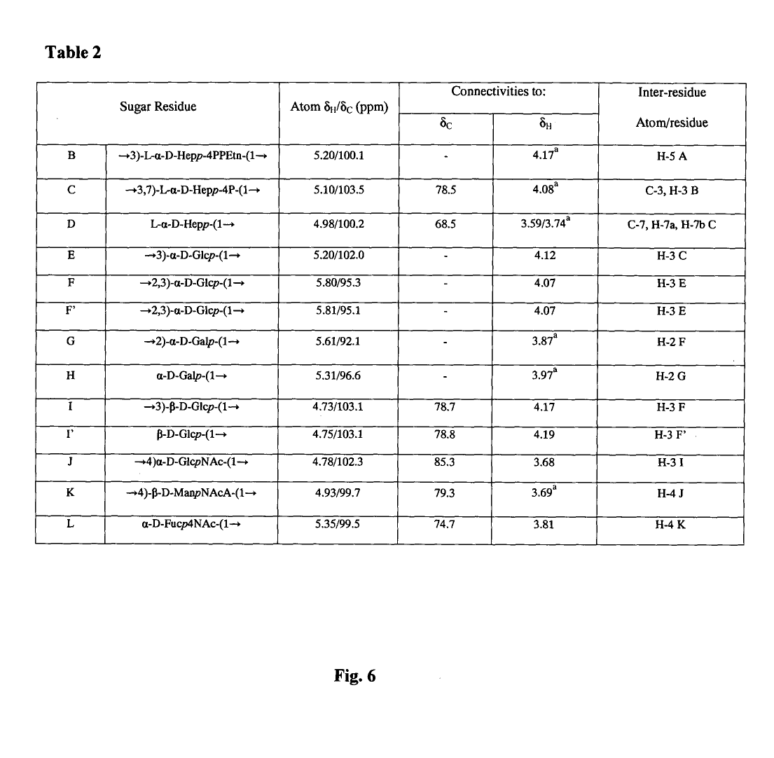

To confirm the results obtained using mass spectrometry and the identification of the substitution site of the core oligosaccharide with the ECA chain, we performed a structural analysis of the fractions of dECA_{LPS} using NMR spectroscopy. The experiments were performed on an Bruker BioSpin Avance 600 apparatus equipped with an Ascend 600 magnet and a CryoProbe QCI. The spectra were recorded at a temperature of 303 K. The sample (1.8 mg) was dissolved in 0.5 ml D₂O. Calibration was performed using acetone as the internal standard. To describe the spin systems of all saccharide residues present in the analysed structure, we performed a series of 2D NMR experiments: ¹H-¹H COSY, TOCSY and ¹H-¹³C HSQC-DEPT. The sequence of particular saccharide residues was performed using ¹H-¹H NOESY and ¹H-¹³C HMBC. The structural analysis showed that the ECA trisaccharide is covalently linked to the core oligosaccharide of Shigella sonnei phase II LOS. The α-D-N-acetylglucosamine residue of the ECA repeating unit is connected by a α(1→6) glycosidic bond to the terminal β-D-glucose of the core oligosaccharide (FORMULA 1, FIG. 4). The chemical shifts of the analysed dECA_{LPS} are shown in Table 1 (FIG. 5). Bonds between the spin systems are presented in Table 2 (FIG. 6) (Gozdziewicz T. K. et al., 2014).

### EXAMPLE 3

### Preparation of Conjugates of dECA_{LPS} with Tetanus Toxoid (dECA_{LPS}-TTd)

The fractions containing core oligosaccharide linked to one, two or three ECA repeating units (dECA_{LPS}) were conjugated with a carrier protein—detoxified tetanus toxin (TTd). To achieve this, oligosaccharides (10 mg) were oxidized with a 10 mM solution of sodium periodate, the reaction was performed for an 1 h at room temperature, in the dark. The mixture was purified using SEC-HPLC on a G3000PW column (Tosoh Bioscience, Japan) equilibrated with water. The oligosaccharide fraction was collected and lyophilised, and then dissolved in 1.5 ml borate buffer (pH=9.0), supplemented with 200 μl of TTd solution (10 mg/ml) and 160 μl 1M sodium cyanoborohydride (ALD, Sterogene, USA). The reaction was conducted at a temperature of 37° C. for 12 days. On days 5 and 10, the reaction mixture was supplemented with subsequent portions of 160 μl ALD. After the reaction was completed, the mixture was purified using SEC-HPLC on a G3000SW column (Tosoh Bioscience) equilibrated with PBS. The fractions containing protein bound to the oligosaccharides were identified using immunoblotting with anti-ECA_{CYC} antibodies. The collected fractions were concentrated on Vivaspin 30 kDa concentrators (Millipore). 0.01% merthiolate was used a preservative.

The concentration of protein in the neoglycoconjugate was determined spectrophotometrically by absorbance measuring at λ=280 nm. About 1 ml of the conjugate at a concentration of 2.02 mg/ml in PBS was obtained. The ratio of dECA_{LPS} to TT in the neoglycoconjugate was determined using mass spectrometry and was 2:1.

### EXAMPLE 4

### Production of Anti-ECA_{LPS}-TTd Serum

In order to evaluate the immunogenicity of the neoglycoconjugate of dECA_{LPS}-dTT, the conjugate was used to immunise rabbits. As the adjuvant, a mixture of a monophosphorylated derivative of Hafnia alveii PCM 1200 lipid A (MPLA), squalene and lecithin was used. The composition of the vaccine dose per rabbit was as follows: 25 μl dECA_{LPS}-TTd conjugate (50 μg), 75 μl MPLA, 200 μl PBS buffer. The mixture was emulsified using ultrasonification, four times for 5 s on ice. T rabbits of the Termond White variety were inoculated. Blood samples were taken prior to immunisation as a control sera. Three subcutaneous injections were administered into rabbit neck (5-6 points), every 21 days. The rabbits were bled 2 weeks after the last dose. The immunization and bleeding procedures were approved by the Local Ethics Committee of IITD PAN (Decision No. 54/2009). The blood was clotted and centrifuged. The resulting sera were heated for 30 min at 56° C. in order to inactivate complement. The sera were stored at −20° C.

### EXAMPLE 5

### Immunogenicity and Cross-Reactivity of Anti-dECA_{LPS}-TT Serum

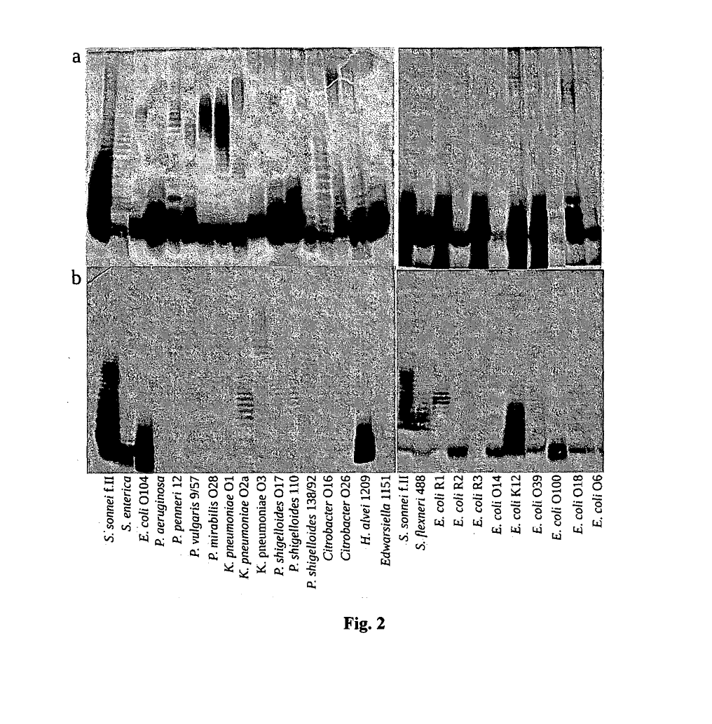

In order to evaluate the reactivity of the resulting polyclonal antibodies with the lipopolysaccharides of a broad group of Gram-negative bacteria, we conducted immunoblotting of selected lipopolysaccharides with the rabbit polyclonal anti-dECA_{LPS}-TTd serum. Lipopolysaccharides were separated electrophoretically on a polyacrylamide gel (FIG. 2a), and then transferred onto a nitrocellulose membrane, which was incubated with a solution of the serum obtained (FIG. 2b). Adsorbed antibodies were detected using anti-IgG antibodies conjugated with horseradish peroxidase.

Anti-dECA_{LPS}-TTd antibodies exhibited a strong, specific reaction with homologous lipooligosaccharides of S. sonnei phase II (rapidly migrating fraction of lipid A substituted with core oligosaccharide and core oligosaccharide substituted with the ECA repeating unit). Additionally, we observed a strong reaction with a slowly migrating polymer, which according to literature data likely corresponds to ECA_{PG} and ECA_{CYC}, contaminants that accompany the preparation of LOS and LPS. The serum also exhibited strong cross-reactions with the rapidly migrating fractions of LPS/LOS (not substituted with O-specific chains) of Salmonella enterica, Hafnia alvei 1209 and E. coli: O104, O100, O14, R2, K12. Weaker cross-reactions were also observed with the rapidly migrating fractions in LPS preparations of E. coli O6, O18 and O39 and Shigella flexneri 488. In the region of slowly migrating fractions weaker cross-reactions were also observed with the LPS of K. pneumoniae O1, O2a, O3, Plesiomonas shigelloides O17, 110, 138/92, Proteus penneri 12, Proteus vulgaris 9/57, Citrobacter O16, E. coli R1, K12, O39, O18, and S. flexneri 488.

### EXAMPLE 6

### Reactivity of the Anti-dECA_{LPS}-TTd Serum Against Live Bacteria

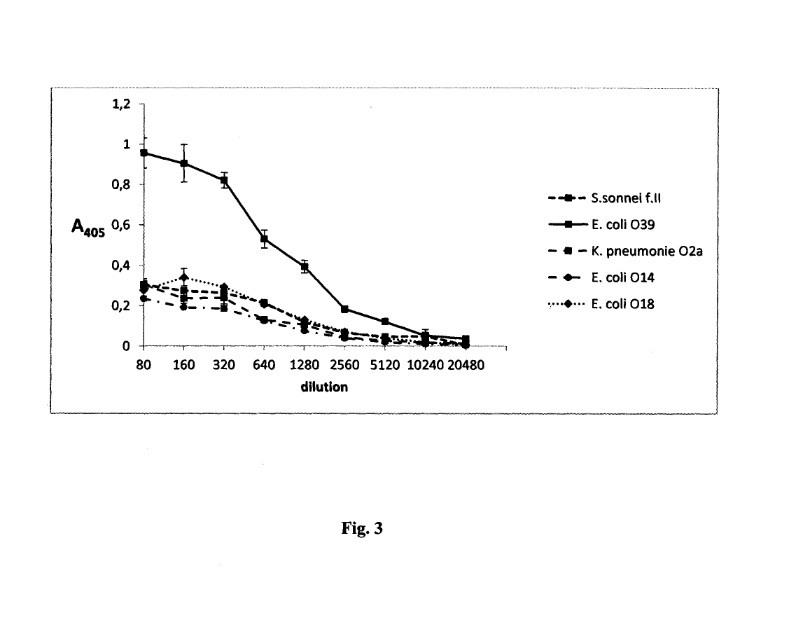

The ability of anti-dECA_{LPS} serum to recognize the ECA antigen on the surface of live bacterial cells was evaluated using an ELISA test and live bacteria as the solid phase in the test (FIG. 3). The negative control was constituted by the serum from the non-immunized rabbit. The goal of the test was to indicate that the access to the epitopes for the obtained antibodies is not hindered by O-specific polysaccharides and capsular antigens. We evaluated the interaction of the serum with the following bacteria: S. sonnei phase II, E. coli O39:K-:H-(PCM 209), E. coli O14:K7(L):H-(PCM 185), E. coli O18ab:K76(B):H14 (PCM 189) and K. pneumoniae O2a. Among the evaluated bacteria there were strains synthesizing the smooth form of LPS and ones lacking the capsular antigen (O39), strains synthesizing the rough form of LPS and lacking the capsular antigen (S. sonnei phase II), strains synthesizing the rough form of LPS and the capsular antigen (O14:K7) and strains synthesizing the smooth form of LPS and capsular antigen (E. coli O18ab:K76 and K. pneumoniae O2a). We observed a positive, but weak reaction of the anti-dECA_{LPS} serum with all strains of bacteria. The strongest reaction was observed against E. coli O39, which synthesizes the smooth form of LPS and lacks the capsular antigen.

### EXAMPLE 7

### Bactericidal Activity of Anti-dECA_{LPS}-TT Serum

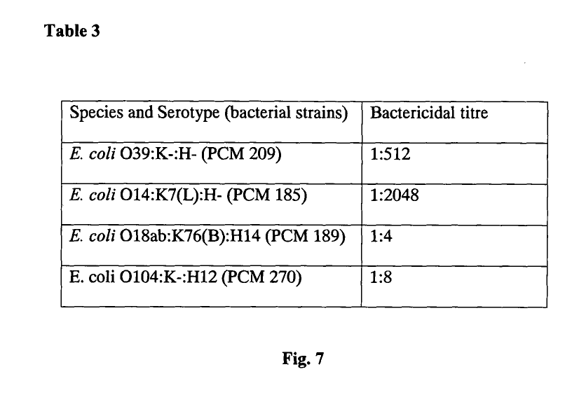

In order to evaluate the protective properties of the obtained anti-dECA_{LPS} serum we determined bactericidal activity against strains of Escherichia coli of following serotypes: O39:K-:H-(PCM 209), O14:K7(L):H-(PCM 185), and O18ab:K76(B):H14 (PCM 189). The bacteria were inoculated and incubated on agar plates at 37° C. overnight, and then suspended in PBS buffer containing 0.1% BSA. The serum was diluted twice in PBS with 0.1% BSA. Rabbit serum was used as a complement source (Biomed Lublin, Poland). Natural antibodies, which may have been present in the serum were absorbed from the complement by incubating serum with fixed bacteria of the evaluated strains (according to K. A. Joiner et al. J. Immunol. 131, 1443, 1983). Appropriately diluted serum in 100 μl was mixed with 80 μl of bacterial suspension and 20 μl of undiluted, absorbed complement. The control was a mixture of 100 μl PBS buffer with 0.1% BSA, 80 μl bacterial suspension and 20 μl complement. The mixtures were incubated at 37° C. for 30 min, and then inoculated onto agar plates (30 μl), incubated overnight and followed by colonies counting. The bactericidal titre was determined to be a serum dilution at which 50% inhibition of colony growth was achieved. The determined bactericidal titres are collected in Table 3 (FIG. 7).

### LITERATURE

Bottger E C, Jurs M, Barrett T, Wachsmuth K, Metzger S, Bitter-Suermann D. 1987. Qualitative and quantitative determination of enterobacterial common antigen (ECA) with monoclonal antibodies: expression of ECA by two Actinobacillus species. J Clin Microbiol. 25:377-382.
Brade L, Hensen S, Brade H. Evaluation of a LPS-based glycoconjugate vaccine against bovine Escherichia coli mastitis: Formation of LPS Abs in cows after immunization with E. coli core oligosaccharides conjugated to hemocyanine. Innate Immun.
Brenner F W, Villar R G, Angulo F J, Tauxe R, Swaminathan B. 2000. Salmonella nomenclature. J Clin Microbiol. 38:2465-2467.
Campbell W N, Hendrix E, Cryz S, Jr., Cross A S. 1996. Immunogenicity of a 24-valent Klebsiella capsular polysaccharide vaccine and an eight-valent Pseudomonas O-polysaccharide conjugate vaccine administered to victims of acute trauma. Clin Infect Dis. 23:179-181.
Cohen D, Ashkenazi S, Green M S, Gdalevich M, Robin G, Slepon R, Yavzori M, Orr N, Block C, Ashkenazi I et al. 1997. Double-blind vaccine-controlled randomised efficacy trial of an investigational Shigella sonnei conjugate vaccine in young adults. Lancet. 349:155-159.
Dell A, Oates J, Lugowski C, Romanowska E, Kenne L, Lindberg B. 1984. The enterobacterial common-antigen, a cyclic polysaccharide. Carbohydr Res. 133:95-104.
Di Padova F E, Brade H, Barclay G R, Poxton I R, Liehl E, Schuetze E, Kocher H P, Ramsay G, Schreier M H, McClelland D B et al. 1993. A broadly cross-protective monoclonal antibody binding to Escherichia coli and Salmonella lipopolysaccharides. Infect Immun. 61:3863-3872.
Di Padova F E, Mikol V, Barclay G R, Poxton I R, Brade H, Rietschel E T. 1994. Anti-lipopolysaccharide core antibodies. Prog Clin Biol Res. 388:85-94.
Duda K A, Duda K T, Beczala A, Kasperkiewicz K, Radziejewska-Lebrecht J, Skurnik M. 2009. ECA-immunogenic of Proteus mirabilis strains. Arch Immunol Ther Exp (Warsz). 57:147-151.
Fregolino E, Ivanova R, Lanzetta R, Molinaro A, Parrilli M, Paunova-Krasteva T, Stoitsova S R, De Castro C. Occurrence and structure of cyclic Enterobacterial Common Antigen in Escherichia coli O157:H(−). Carbohydr Res. 363:29-32.
Gozdziewicz T. K., Lugowski C., Lukasiewicz J. 2014. First evidence for a covalent linkage between enterobacterial common antigen and lipopolysaccharide in Shigella sonnei phase II ECA_{LPS}. Journal of Biological Chemistry, 289, 2745-2754.
Grandjean C, Wade T K, Ropartz D, Ernst L, Wade W F. 2013. Acid-detoxified Inaba lipopolysaccharide (pmLPS) is a superior cholera conjugate vaccine immunogenic than hydrazine-detoxified lipopolysaccharide and induces vibriocidal and protective antibodies. Pathogens Dis. 67:136-158.
Gupta R K, Szu S C, Finkelstein R A, Robbins J B. 1992. Synthesis, characterization, and some immunological properties of conjugates composed of the detoxified lipopolysaccharide of Vibrio cholerae O1 serotypes Inaba bound to cholera toxin. Infect Immun. 60:3201-3208.
Hermanson G T. 2008. Bioconjugate techniques. 2nd ed. Elsevier:Rockford.
Kiss P, Rinno J, Schmidt G, Mayer H. 1978. Structural studies on the immunogenic form of the enterobacterial common antigen. Eur J Biochem. 88:211-218.
Kossaczka Z, Shiloach J, Johnson V, Taylor D N, Finkelstein R A, Robbins J B, Szu S C. 2000. Vibrio cholerae O139 conjugate vaccines: synthesis and immunogenic of V. cholerae O139 capsular polysaccharide conjugates with recombinant diphtheria toxin mutants in mice. Infect Immun. 68:5037-5043.
Kuhn H M, Meier-Dieter U, Mayer H. 1988. ECA, the enterobacterial common antigen. FEMS Microbiol Rev. 4:195-222.
Kuhn H M, Neter E, Mayer H. 1983. Modification of the lipid moiety of the enterobacterial common antigen by the “Pseudomonas factor”. Infect Immun. 40:696-700.
Kunin C M. 1963. SEPARATION, CHARACTERIZATION, AND BIOLOGICAL SIGNIFICANCE OF A COMMON ANTIGEN IN ENTEROBACTERIACEAE. J Exp Med. 118:565-586.
Kunin C M, Beard M V. 1963. SEROLOGICAL STUDIES OF O ANTIGENS OF ESCHERICHIA COLI BY MEANS OF THE HEMAGGLUTINATION TEST. J Bacteriol. 85:541-548.
Kurupati P, Teh B K, Kumarasinghe G, Poh C L. 2006. Identification of vaccine candidate antigens of an ESBL producing Klebsiella pneumoniae clinical strain by immunoproteome analysis. Proteomics. 6:836-844.
Lugowski C. 1987. [Structure and immunological properties of enterobacterial common antigen (ECA)]. Postepy Hig Med Dosw. 41:384-432.
Lugowski C, Jachymek W, Niedziela T, Rowinski S. 1996a. Serological characterisation of anti-endotoxin sera directed against the conjugates of oligosaccharide core of Escherichia coli type R1, R2, R3, J5 and Salmonella Ra with tetanus toxoid. FEMS Immunol. Med. Microbiol. 16:21-30.
Lugowski C, Kulakowska M, Romanowska E. 1983. Enterobacterial common antigen-tetanus toxoid conjugate as immunogenic. Infect Immun. 42:1086-1091.
Lugowski C, Niedziela T, Jachymek W. 1996b. Anti-endotoxin antibodies directed against Escherichia coli R-1 oligosaccharide core-tetanus toxoid conjugate bind to smooth, live bacteria and smooth lipopolysaccharides and attenuate their tumor necrosis factor stimulating activity. FEMS Immunol. Med. Microbiol. 16:31-38.
Lugowski C, Romanowska E. 1978. Enterobacterial common antigen: isolation from Shigella sonnei, purification and immunochemical characterization. Eur J Biochem. 91:89-97.
Lugowski C, Romanowska E. 1991. Characterization of an enterobacterial common antigen (ECA) epitopes recognized by anti-ECA-tetanus toxoid conjugate serum. FEMS Microbiol Lett. 61:315-318.
Lukasiewicz J, Jachymek W, Niedziela T, Malik-Gebicka M, Dzieciatkowska M, Lugowski C. 2002. Comparison of serological specificity of anti-endotoxin sera directed against whole bacterial cells and core oligosaccharide of Escherichia coli J5-tetanus toxoid conjugate. Acta Biochim. Pol. 49:721-734.
Lukasiewicz J, Lugowski C. 2003. [Biologic activity of lipopolysaccharides]. Postepy Hig Med Dosw. 57:33-53.
Lugowski C, Romanowska E. 1978. Enterobacterial common antigen: isolation from Shigella sonnei, purification and immunochemical characterization. Eur. J. Biochem. 91:89-97.
McIntyre P B, O'Brien K L, Greenwood B, van de Beek D. Effect of vaccines on bacterial meningitis worldwide. Lancet. 380:1703-1711.
Mendoza N, Ravanfar P, Satyaprakash A, Pillai S, Creed R. 2009. Existing antibacterial vaccines. Dermatol Ther. 22:129-142.
Muller-Loennies S, Brade L, Brade H. 2007. Neutralizing and cross-reactive antibodies against enterobacterial lipopolysaccharide. Int J Med Microbiol. 297:321-340.
Niedziela T, Letowska I, Lukasiewicz J, Kaszowska M, Czamecka A, Kenne L, Lugowski C. 2005. Epitope of the Vaccine-Type Bordetella pertussis Strain 186 Lipooligosaccharide and Antiendotoxin Activity of Antibodies Directed against the Terminal Pentasaccharide-Tetanus Toxoid Conjugate. Infect. Immun. 73:7381-7389.
Olsthoorn M M, Petersen B O, Schlecht S, Haverkamp J, Bock K, Thomas-Oates J E, Hoist O. 1998. Identification of a novel core type in Salmonella lipopolysaccharide. Complete structural analysis of the core region of the lipopolysaccharide from Salmonella enterica sv. Arizonae O62. J Biol Chem. 273:3817-3829.
Peters H, Jurs M, Jann B, Jann K, Timmis K N, Bitter-Suermann D. 1985. Monoclonal antibodies to enterobacterial common antigen and to Escherichia coli lipopolysaccharide outer core: demonstration of an antigenic determinant shared by enterobacterial common antigen and E. coli K5 capsular polysaccharide. Infect Immun. 50:459-466.
Phalipon A, Tanguy M, Grandjean C, Guerreiro C, Belot F, Cohen D, Sansonetti P J, Mulard L A. 2009. A synthetic carbohydrate-protein conjugate vaccine candidate against Shigella flexneri 2a infection. J Immunol. 182:2241-2247.
Pozsgay V, Kubler-Kielb J, Schneerson R, Robbins J B. 2007. Effect of the nonreducing end of Shigella dysenteriae type 1 O-specific oligosaccharides on their immunogenic as conjugates in mice. Proc Natl Acad Sci USA. 104:14478-14482.
Radziejewska-Lebrecht J, Skurnik M, Shashkov A S, Brade L, Rozalski A, Bartodziejska B, Mayer H. 1998. Immunochemical studies on R mutants of Yersinia enterocolitica O:3. Acta Biochim Pol. 45:1011-1019.
Rick P D, Hubbard G L, Kitaoka M, Nagaki H, Kinoshita T, Dowd S, Simplaceanu V, Ho C. 1998. Characterization of the lipid-carrier involved in the synthesis of enterobacterial common antigen (ECA) and identification of a novel phosphoglyceride in a mutants of Salmonella typhimurium defective in ECA synthesis. Glycobiology. 8:557-567.
Rietschel E T, Brade H, Hoist O, Brade L, al. e. 1996. Bacterial endotoxin: chemical constitution, biological recognition, host response, and immunological detoxification. In: Rietschel E T, Wagner H editors. Pathology of Septic Shock ed. Berlin, Heidelberg, N.Y.: Springer-Verlag. p. 40-81.
Robbins J B, Kubler-Kielb J, Vinogradov E, Mocca C, Pozsgay V, Shiloach J, Schneerson R. 2009. Synthesis, characterization, and immunogenic in mice of Shigella sonnei O-specific oligosaccharide-core-protein conjugates. Proc Natl Acad Sci USA. 106:7974-7978.
Romanowska E, Katzenellenbogen E, Lugowski C, Gamian A, Bogulska M. 1978. Immunochemical characteristics of Shigella sonnei and serotypes 6 Shigella flexneri lipopolysaccharides and enterobacterial common antigen. Arch. Immunol. Ther. Exp. (Warsz). 26:249-254.
Swartley J S, Marfin A A, Edupuganti S, Liu L J, Cieslak P, Perkins B, Wenger J D, Stephens D S. 1997. Capsule switching of Neisseria meningitidis. Proc Natl Acad Sci USA. 94:271-276.
Weintraub A. 2003. Immunology of bacterial polysaccharide antigens. Carbohydr Res. 338:2539-2547.
Whitfield C. 2006. Biosynthesis and assembly of capsular polysaccharides in Escherichia coli. Annu Rev Biochem. 75:39-68.
Woodford N, Turton J F, Livermore D M. Multiresistant Gram-negative bacteria: the role of high-risk clones in the dissemination of antibiotic resistance. FEMS Microbiol Rev. 35:736-755.
Wyres K L, Lambertsen L M, Croucher N J, McGee L, von Gottberg A, Li\check{A}±ares J, Jacobs M R, Kristinsson K G, Beall B W, Klugman K P et al. 2013. Pneumococcal Capsular Switching: A Historical Perspective. Journal of Infectious Diseases. 207:439-449.

1-20. (canceled)

21. An immunogenic antigen comprising enterobacterial common antigen ECA repeating unit of the structure [→3)-α-D-Fuc4NAc-(1→4)-β-D-ManNAcA-(1→4)-α-D-GlcNAc-(1→]ₙ, wherein n≧1, covalently linked to a core oligosaccharide originating from a lipopolysaccharide LPS or lipooligosaccharide LOS of Gram-negative bacteria.

22. An immunogenic antigen according to claim 21, characterized in that the ECA repeating unit is linked with the core oligosaccharide by a glycosidic bond.

23. An immunogenic antigen according to claim 21, characterized in that the core oligosaccharide originates from LPS or LOS of E. coli, Salmonella, Shigella, Klebsiella, Edwarsiella, Enterobacter, Citrobacter, Serratia, Proteus, Yersinia, Erwinia, Plesiomonas, Aeromonas.

24. An immunogenic antigen according to claim 23, characterized in that the core oligosaccharide originates from LOS of S. sonnei phase II.

25. An immunogenic antigen according to claim 21, characterized in that it originates from Shigella sonnei phase II and has the following structure of the enterobacterial common antigen (ECA) repeating unit—core oligosaccharide region:

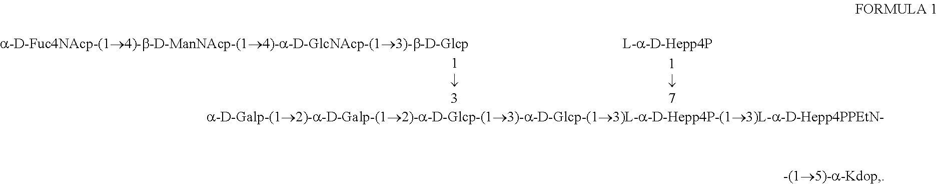

26. A pharmaceutical composition comprising the antigen as defined in claim 21, and a pharmaceutically permissible carrier.

27. A pharmaceutical composition as defined in claim 26 for use in the treatment of diseases caused by Gram-negative bacteria, preferably of the Enterobacteriacea family.

28. A pharmaceutical composition for use in the treatment of diseases caused by Gram-negative bacteria according to claim 27, characterized in that the disease is selected from a group encompassing nosocomial infections and septic shock and enteritis.

29. A pharmaceutical composition for use in the treatment of diseases caused by Gram-negative bacteria according to claim 27, characterized in that the disease is caused by bacteria selected from the group comprising Shigella spp., Salmonella spp., Klebsiella spp., Serratia spp., Enterobacter and E. coli (ETEC, EPEC, EHEC, EIEC, EAEC, UPEC, MNEC).

30. A bactericidal antibody that binds epitopes of the antigen as defined in claim 21 and cross-reacts with strains of Gram-negative bacteria, particularly with bacteria of the family Enterobacteriaceae

31. A bactericidal antibody according to claim 30, characterized in that the cross-reaction occurs with strains of bacteria selected from a group encompassing S. sonnei phase II, S. enteritica, E. coli (O39, O100, O18, O6), H. alvei and with ECA_{PG} S. sonnei phase II, S. enteritica, E. coli, Proteus vulgaris, P. mirabilis, K. pneumoniae, P. shigelloides, Citrobacter.

32. A glycoconjugate comprising the immunogenic antigen as defined in claim 21, and a carrier protein.

33. A glycoconjugate according to claim 32, characterised in that the carrier protein is selected from a group encompassing tetanus toxin or toxoid (TT/TTd), diphtheria toxin or toxoid (DT/DTd), diphtheria toxin mutant CRM197, exotoxin A Pseudomonas, toxin B or toxoid of Clostridium difficile, cholera toxin or toxoid (TC/TCd), toxins of Streptococcus group A, pneumolysin of Streptococcus pneumoneae, filamentous haemagglutinin (FHA) Bordetella pertussis and fragments thereof, pile and pilin of Neisseria gonorrhoeae, outer membrane protein (OMP).

34. A glycoconjugate according to claim 33, characterized in that the carrier protein is selected from a group comprising tetanus toxin or toxoid (TT/TTd).

35. A vaccine containing a glycoconjugate as defined in claim 32, and a pharmaceutically permissible carrier and optionally an adjuvant.
